(19) United States
(12) Patent Application Publication
(10) Pub. No.: US 2010/0152848 A1
(43) Pub. Date: Jun. 17, 2010

(54) ARTIFICIAL INTRAOCULAR LENS, ALTERED NATURAL CRYSTALLINE LENS, OR REFILLED NATURAL CRYSTALLINE LENS CAPSULE WITH ONE OR MORE SCLERAL PROSTHESES FOR IMPROVED PERFORMANCE

(75) Inventors: Douglas C. Williamson, Coppell, TX (US); Barrie D. Soloway, Long Beach, NY (US); Gary A. Richardson, Rochester, NY (US)

Correspondence Address: DOCKET CLERK, P.O. DRAWER 800889, DALLAS, TX, 75380, US

(73) Assignee: Refocus Group, Inc., Dallas, TX (US)

(21) Appl. No.: 12/621,699

(22) Filed: Nov. 19, 2009

Related U.S. Application Data
(60) Provisional application No. 61/199,726, filed on Nov. 19, 2008.

(51) Int. Cl. A61F 2/16

(52) U.S. Cl. 623/6.11; 623/4.1

(57) ABSTRACT

A system includes an intraocular lens configured to replace a natural crystalline lens of an eye. The system also includes one or more scleral prostheses configured to be inserted into scleral tissue of the eye. The one or more scleral prostheses are configured to modify a structure of the eye to improve an accommodative ability of the eye with the intraocular lens. The intraocular lens could represent an accommodating intraocular lens, and the one or more scleral prostheses could be configured to increase an amount of accommodation achievable using the accommodating intraocular lens. The intraocular lens could also represent a non-accommodating intraocular lens, and the one or more scleral prostheses could be configured to provide an amount of accommodation achievable using the non-accommodating intraocular lens.

### CROSS-REFERENCE TO RELATED PATENT DOCUMENTS AND PRIORITY CLAIM

This application claims priority under 35 U.S.C. §119(e) to U.S. Provisional Patent Application No. 61/199,726 filed on Nov. 19, 2008, which is hereby incorporated by reference.

This application is related to the following U.S. patent documents:
(1) U.S. Pat. No. 6,007,578 entitled “Scleral Prosthesis for Treatment of Presbyopia and Other Eye Disorders” issued on Dec. 28, 1999;
(2) U.S. Pat. No. 6,280,468 entitled “Scleral Prosthesis for Treatment of Presbyopia and Other Eye Disorders” issued on Aug. 28, 2001;
(3) U.S. Pat. No. 6,299,640 entitled “Scleral Prosthesis for Treatment of Presbyopia and Other Eye Disorders” issued on Oct. 9, 2001;
(4) U.S. Pat. No. 5,354,331 entitled “Treatment of Presbyopia and Other Eye Disorders” issued on Oct. 11, 1994;
(5) U.S. Pat. No. 5,465,737 entitled “Treatment of Presbyopia and Other Eye Disorders” issued on Nov. 14, 1995;
(6) U.S. Pat. No. 5,489,299 entitled “Treatment of Presbyopia and Other Eye Disorders” issued on Feb. 6, 1996;
(7) U.S. Pat. No. 5,503,165 entitled “Treatment of Presbyopia and Other Eye Disorders” issued on Apr. 2, 1996;
(8) U.S. Pat. No. 5,529,076 entitled “Treatment of Presbyopia and Other Eye Disorders” issued on Jun. 25, 1996;
(9) U.S. Pat. No. 5,722,952 entitled “Treatment of Presbyopia and Other Eye Disorders” issued on Mar. 3, 1998;
(10) U.S. Pat. No. 6,197,056 entitled “Segmented Scleral Band for Treatment of Presbyopia and Other Eye Disorders” issued on Mar. 6, 2001;
(11) U.S. Pat. No. 6,579,316 entitled “Segmented Scleral Band for Treatment of Presbyopia and Other Eye Disorders” issued on Jun. 17, 2003;
(12) U.S. Pat. No. 6,926,727 entitled “Surgical Blade for Use with a Surgical Tool for Making Incisions for Scleral Eye Implants” issued on Aug. 9, 2005;
(13) U.S. Pat. No. 6,991,650 entitled “Scleral Expansion Device Having Duck Bill” issued on Jan. 31, 2006;
(14) U.S. patent application Ser. No. 10/080,877 entitled “System and Method for Making Incisions for Scleral Eye Implants” filed on Feb. 22, 2002;
(15) U.S. patent application Ser. No. 10/443,122 entitled “System and Method for Determining a Position for a Scleral Pocket for a Scleral Prosthesis” filed on May 20, 2003;
(16) U.S. patent application Ser. No. 11/137,085 entitled “Scleral Prosthesis for Treatment of Presbyopia and Other Eye Disorders” filed on May 24, 2005;
(17) U.S. patent application Ser. No. 11/199,591 entitled “Surgical Blade for Use with a Surgical Tool for Making Incisions for Scleral Eye Implants” filed on Aug. 8, 2005;
(18) U.S. patent application Ser. No. 11/252,369 entitled “Scleral Expansion Device Having Duck Bill” filed on Oct. 17, 2005;
(19) U.S. patent application Ser. No. 11/323,283 entitled “Surgical Blade for Use with a Surgical Tool for Making Incisions for Scleral Eye Implants” filed on Dec. 30, 2005;
(20) U.S. patent application Ser. No. 11/323,284 entitled “System and Method for Making Incisions for Scleral Eye Implants” filed on Dec. 30, 2005;
(21) U.S. patent application Ser. No. 11/322,728 entitled “Segmented Scleral Band for Treatment of Presbyopia and Other Eye Disorders” filed on Dec. 30, 2005;
(22) U.S. patent application Ser. No. 11/323,752 entitled “Segmented Scleral Band for Treatment of Presbyopia and Other Eye Disorders” filed on Dec. 30, 2005;
(23) U.S. Provisional Patent Application No. 60/819,995 entitled “Apparatuses, Systems, and Methods Related to Treating Presbyopia and Other Eye Disorders” filed on Jul. 11, 2006;
(24) U.S. patent application Ser. No. 11/827,444 entitled “Apparatus and Method for Securing Ocular Tissue” filed on Jul. 11, 2007;
(25) U.S. patent application Ser. No. 11/827,382 entitled “Scleral Prosthesis for Treating Presbyopia and Other Eye Disorders and Related Devices and Methods” filed on Jul. 11, 2007;
(26) U.S. Provisional Patent Application No. 61/001,593 entitled “Apparatuses and Methods for Forming Incisions in Ocular Tissue” filed on Nov. 2, 2007;
(27) U.S. Provisional Patent Application No. 61/065,149 entitled “Scleral Prosthesis for Ocular Drug Delivery to Treat Glaucoma, Macular Degeneration, and Other Eye Disorders or Diseases and Related Method” filed on Feb. 8, 2008; and
(28) U.S. Provisional Patent Application No. 61/072,757 entitled “System and Method for Identifying a Position to Insert a Scleral Prosthesis into an Eye” filed on Apr. 2, 2008.
   All of these patents and patent applications are hereby incorporated by reference.

### TECHNICAL FIELD

This disclosure is generally directed to ocular devices. More specifically, this disclosure is directed to an artificial intraocular lens, altered natural crystalline lens, or refilled natural crystalline lens capsule (or other intraocular lens implant) with one or more scleral prostheses for improved performance.

### BACKGROUND

The natural crystalline lens of the eye may need alteration or replacement for any number of reasons. These reasons include, but are not limited to, opacification of the lens (causing cataract) or natural aging of the lens (causing presbyopia). Often times, these or other problems may require removal of the natural crystalline lens and replacement with an artificial intraocular lens (IOL) during a surgical eye procedure.

There are various types of intraocular lenses on the market today, including “accommodating” and “non-accommodating” lenses. “Accommodation” in this sense refers to the ability of the eye to dynamically focus on near objects, providing a range of multiple near focal points. The range of multiple focal points in a young person is provided by the crystalline lens, which changes shape in order to see various objects at near. However, as a person ages, the range of near focal points gradually diminishes, and the ability to see at near is typically diminished significantly by the age of 45 (a condition known as presbyopia).

Accommodating intraocular lenses typically provide (or claim to provide) a small amount of accommodation, allowing a patient to focus on more than one near focal point in a manner similar to that of a person 30 to 40 years old. However, in many existing accommodating intraocular lenses, the range of near focal points may be quite limited.

Non-accommodating intraocular lenses may be monofocal, having one fixed focal point that can be at distance or at near as determined by the prescription of the lens and provide no dynamic accommodation abilities. Another type of non-accommodating intraocular lens has multiple fixed focal points (typically one at distance and one at near), which are provided using non-spheric or diffractive optics. These are typically classified as multi-focal intraocular lenses.

Other techniques to alter the natural crystalline lens may also be used to treat lens disorders. These techniques could include the application of pharmaceutical agents to the lens. These techniques could also include the use of (i) laser, other light, or other electro-magnetic radiation and/or (ii) sound or ultrasound waves. These techniques could further include the removal and replacement of part or all of the lens material with a refilling type procedure.

It is also possible to treat presbyopia, glaucoma, and other eye disorders by implanting scleral prostheses within the sclera of a patient's eye. For each individual scleral prosthesis, an incision is made in the sclera of the patient's eye. The incision is then extended under the surface of the sclera to form a scleral “tunnel,” and a scleral prosthesis is placed within the tunnel. One or multiple scleral prostheses may be implanted in a patient's eye to (among other things) treat presbyopia, glaucoma, ocular hypertension, elevated intraocular pressure, or other eye disorders. This technique is described more fully in the related U.S. patents documents incorporated by reference above.

### SUMMARY

This disclosure provides an artificial intraocular lens, altered natural crystalline lens, or refilled natural crystalline lens capsule (or other intraocular lens implant) with one or more scleral prostheses for improved performance.

In a first embodiment, a system includes an intraocular lens configured to replace a natural crystalline lens of an eye. The system also includes one or more scleral prostheses configured to be inserted into scleral tissue of the eye. The one or more scleral prostheses are configured to modify a structure of the eye to improve an accommodative ability of the eye with the intraocular lens.

In a second embodiment, a method includes inserting an intraocular lens into an eye to replace a natural crystalline lens of the eye. The method also includes inserting one or more scleral prostheses into scleral tissue of the eye. The one or more scleral prostheses modify a structure of the eye and improve an accommodative ability of the eye with the intraocular lens.

In a third embodiment, a method includes modifying a natural lens of an eye and inserting one or more scleral prostheses into scleral tissue of the eye. The one or more scleral prostheses modify a structure of the eye to improve an accommodative ability of the eye with the modified natural lens.

In a fourth embodiment, a method includes filling a crystalline lens capsule of an eye with one or more materials. The method also includes inserting one or more scleral prostheses into scleral tissue of the eye. The one or more scleral prostheses modify a structure of the eye to improve an accommodative ability of the eye with the filled crystalline lens capsule.

Other technical features may be readily apparent to one skilled in the art from the following figures, descriptions, and claims.

### BRIEF DESCRIPTION OF THE DRAWINGS

For a more complete understanding of this disclosure and its features, reference is now made to the following description, taken in conjunction with the accompanying drawings, in which:

FIGS. 1 and 2 illustrate an example eye in a presbyopic person focusing at distance without accommodation and attempting to focus at near without modification;

FIGS. 3 and 4 illustrate an example eye in a presbyopic person focusing at distance and focusing at near with modification;

FIGS. 5 and 6 illustrate an example eye having a non-accommodating intraocular lens focusing at distance and focusing at near with modification;

FIGS. 7 and 8 illustrate an example eye having an accommodating intraocular lens focusing at distance and focusing at near with modification;

FIGS. 9 and 10 illustrate an example eye having another accommodating intraocular lens focusing at distance and focusing at near with modification; and

FIG. 11 illustrates an example method for providing improved accommodation in an eye.

### DETAILED DESCRIPTION

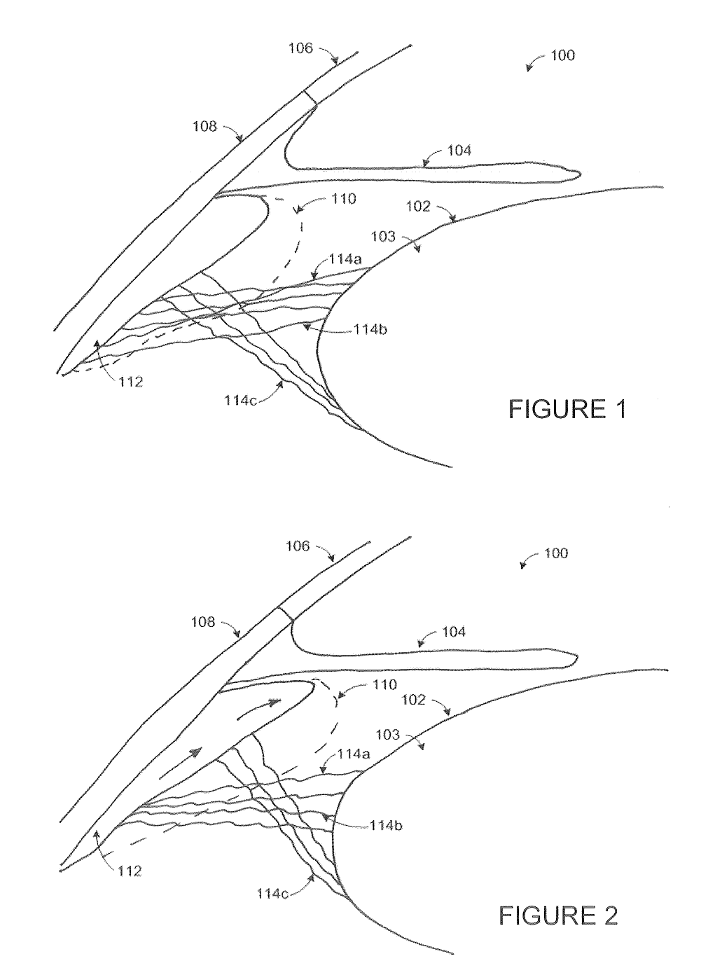

FIGS. 1 through 11, discussed below, and the various embodiments used to describe the principles of the present invention in this patent document are by way of illustration only and should not be construed in any way to limit the scope of the invention. Those skilled in the art will understand that the principles of the present invention may be implemented in any type of suitably arranged device or system.

In accordance with this disclosure, an artificial intraocular lens, altered natural crystalline lens, or refilled natural crystalline lens capsule and one or more scleral prostheses can be used in a patient's eye. For example, an artificial intraocular lens and one or more scleral prostheses could be inserted into the patient's eye at the same time or at different times. Also, the natural crystalline lens may or may not be altered, or the natural crystalline lens capsule may or may not be refilled, at the same time that the one or more scleral prostheses are inserted into the patient's eye. The artificial intraocular lens, altered natural crystalline lens, or refilled natural crystalline lens capsule could be accommodating or non-accommodating. The one or more scleral prostheses could (i) improve the accommodative abilities of an accommodating intraocular lens, (ii) provide accommodation for a non-accommodating intraocular lens, or (iii) improve or provide an accommodating effect to an altered natural crystalline lens or to a refilled natural crystalline lens capsule. Any suitable lens alteration technique or lens capsule refilling technique could be used here, or any suitable intraocular lens could be used here. Similarly, any suitable scleral prosthesis could be used here, such as any of the scleral prostheses disclosed in the U.S. patent documents incorporated by reference above.

FIGS. 1 and 2 illustrate an example eye 100 in a presbyopic person focusing at distance without accommodation and attempting to focus at near without modification. In particular, FIG. 1 illustrates the eye 100 in the presbyopic person focusing at distance without accommodation, and FIG. 2 illustrates an example of the eye 100 in a presbyopic person attempting to focus at near without modification.

As shown in FIG. 1, the eye 100 includes a crystalline lens capsule 102, a crystalline lens 103, an iris 104, a cornea 106, and a sclera 108. In general, the crystalline lens 103 focuses light entering the eye 100 through the cornea 106 on the retina at the back of the eye. The sclera 108 represents the tough outer white portion of the eye 100. The eye 100 also includes the ciliary processes 110 and the ciliary muscles 112 (collective called the “ciliary body”). The ciliary processes 110 include soft glands connected to the outer surface of the ciliary muscles 112. The ciliary processes 110 produce aqueous, which is constantly flowing across the anterior surface of the crystalline lens 103, up through the pupil of the eye, and out through a series of pores at the outer edge of the iris 104 called the trabecular meshwork. The aqueous provides nourishment for the crystalline lens 103 and the cornea 106 and provides pressure for the eye 100. The ciliary muscles 112 are attached to the inner surface of the sclera 108. The crystalline lens 103 is held within a thin flexible envelope made of tissue known as the crystalline lens capsule 102.

The ciliary muscles 112 are attached to the crystalline lens capsule 102 by various fibers known as zonules 114a-114c, which pass through the ciliary processes 110 before reaching the lens capsule 102. In response to changes in the position of the ciliary muscles 112, the zonules 114a-114c can manipulate the lens capsule 102, causing the crystalline lens 103 to change shape and become more convex. When the lens 103 becomes more convex, its refractive power increases, changing how rays of light fall on the retina and allowing the eye 100 to focus at near. However, as the eye ages, the working distance between the outer diameter of the crystalline lens 103 and the ciliary muscles 112 diminishes. This eventually causes relaxation in the tension that the zonules 114a-114c can exert on the crystalline lens 103. For most people (typically by the age of 45), the loss of tension on the zonules becomes great enough that changes in the position of the ciliary muscles 112 can no longer adequately change the shape of the crystalline lens 103 for near vision without additional optical correction. By age 65, most people lose the ability to focus at near altogether. The eye 100 shown in FIG. 1 is the eye of a person who is suffering from presbyopia, and the zonules 114a-114c no longer exert enough tension to change the shape of the crystalline lens 103 to permit dynamic accommodation. This lack of tension in the zonules is portrayed graphically by “waves” in the path between the attachment points for each zonule.

The zonules 114a-114c here include anterior zonules 114a, equatorial zonules 114b, and posterior zonules 114c (which are based on where the zonules connect to the crystalline lens capsule 102). In general, the anterior zonules 114a typically connect to the lens capsule 102 approximately 1.5-2.0 mm anterior to the equatorial plane of the crystalline lens 103. The equatorial zonules 114b typically connect to the lens capsule 102 at approximately the lens equator itself. The posterior zonules 114c typically connect to the lens capsule 102 approximately 1.5-2.0 mm posterior to the lens equator.

As depicted in FIG. 1, the anterior, equatorial and posterior zonules 114a-114c criss-cross before attaching to the ciliary muscles 112 so that the posterior zonules 114c are attached to a point anterior to both the equatorial and anterior zonules 114a-114b. In FIG. 1, the crystalline lens 103 is in its relaxed state, and the ciliary muscles 112 are similarly in their relaxed non-accommodated state, meaning the lens 103 is focused at distance (focused for distance viewing).

As shown in FIG. 2, the eye 100 is attempting to focus at near (such as on a near object), and the ciliary muscles 112 contract. Due to the ring-like shape of the ciliary muscles 112 as they encircle the inside of the sclera 108, this contraction causes its mass to move inward and upward to a position of a smaller minor circle on the interior of the globe. This movement moves the attachment points for the zonules 114a-114c on the ciliary muscles 112 upwards as well. In a young person with a natural crystalline lens 103 and without presbyopia, this movement affects the shape of the natural crystalline lens 103, allowing the lens 103 to increase its dioptric focusing power and become focused at near. However, the upward and inward movement of the ciliary muscles 112 in a person who has presbyopia (as depicted in FIG. 2) does not result in a change in the shape of the natural crystalline lens 103, so there is little or no increase in the dioptric focusing power of the lens. This is primarily because the tension on the zonules 114a-114c decreases as a person ages, which may be due to the outward growth of the crystalline lens 103 and/or the inward growth of the ciliary body/muscles towards the lens 103. As a result, the “circumlenticular” distance between the ciliary body/muscles and the crystalline lens 103 is reduced on a linear basis with age, reducing the tension on at least some of the zonules 114a-114c (shown here by the “wavy” lines representing loose or slack zonules) until there is no longer enough tension to change the shape of the lens 103. Because of this, even though the ciliary body/muscles are still contracting, a person (typically starting at the age of 45) often loses the ability to focus on near objects, and is thus said to have the condition known as presbyopia.

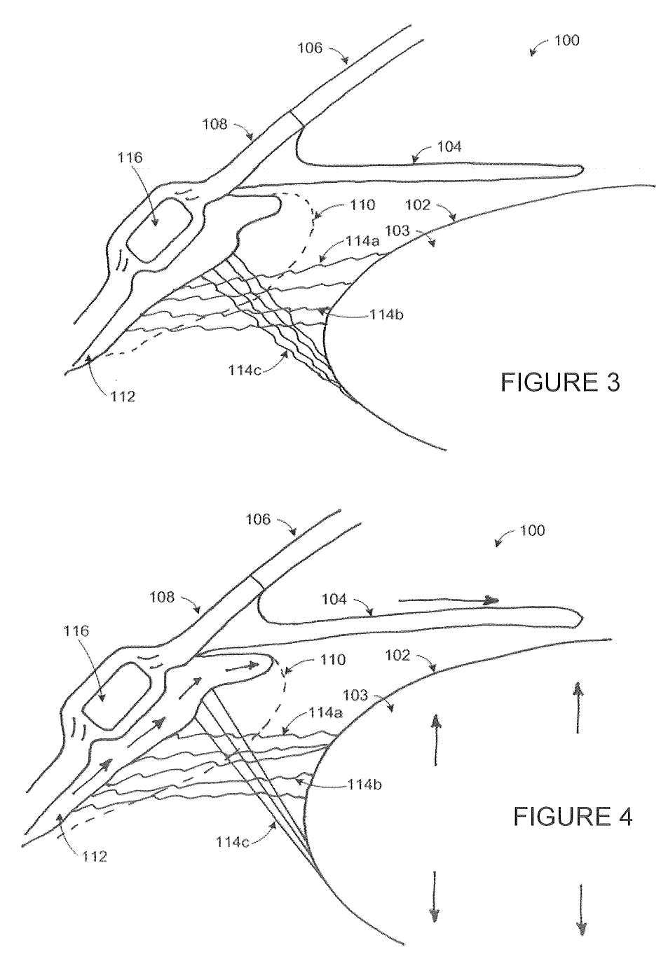

FIGS. 3 and 4 illustrate an example eye in a presbyopic person focusing at distance and focusing at near with modification. As noted in the U.S. patent documents incorporated by reference above, one or more scleral prostheses can be used to help reduce or eliminate presbyopia (as well as other eye disorders). FIG. 3 illustrates an example of the eye 100 focusing at distance with modification, and FIG. 4 illustrates an example of the eye 100 focusing at near with modification.

As shown in FIG. 3, a scleral prosthesis 116 has been inserted into the patient's scleral tissue. The patient illustrated here is presbyopic, which is depicted with loose or “wavy” zonules 114a-114c due to the reduced distance between the edge of the crystalline lens 103 and the ciliary body/muscles. The scleral prosthesis 116 creates “vaulting” at its anterior surface and/or its posterior surface. This may or may not immediately cause tightening of at least some of the zonules 114a-114c, depending upon the exact attachment points of the zonules on the ciliary muscles 112 at rest. In FIG. 3, the scleral prosthesis 116 is shown as having no immediate effect on tightening the zonules because (i) the attachment points for the zonules are shown as being below the point of vaulting from the scleral prosthesis 116 and (ii) the eye 100 is focused at distance.

As shown in FIG. 4, the ciliary muscles 112 contract, moving upward and inward. This moves the attachment points for the zonules 114a-114c upwards as well. However, in this case the vaulting created by the sclera prosthesis 116 exaggerates the tension on at least some of the zonules 114a-114c, actually restoring the tension experienced during a patient's younger years (also known as restoring the “working distance” between the lens 103 and the ciliary muscles 112). This increased tension causes the shape of the lens 103 to “round-up” or become more convex centrally, thereby changing the dioptric power of the lens 103 and allowing the patient to focus on near objects. This helps to reduce or eliminate presbyopia in the patient.

The Helmholtz theory of presbyopia postulates that the movement of the ciliary muscles (or the ciliary body) is mostly inward directly towards the center of the crystalline lens 103, releasing tension on all zonules evenly and allowing the crystalline lens 103 to “round-up” during accommodation. However, recent research indicates that the ciliary muscles 112 move both upward and inward during accommodation (during focusing on near objects) to a smaller minor circle of the globe of the eye, which is illustrated in FIGS. 1 through 4. Regardless of the movement of the ciliary muscles 112 during accommodation, it has been established that the insertion of one or more scleral prostheses 116 into a patient's eye can help to restore accommodative power to the crystalline lens 103 of the eye.

Moreover, in FIGS. 1 through 4, the anterior zonules 114a and the posterior zonules 114c are shown to “criss-cross.” Again, recent research indicates that the anterior and posterior zonules may criss-cross either within the ciliary processes 110 or possibly even prior to entering the ciliary processes 110. With this configuration of zonules, the accommodation experienced in FIG. 4 can be explained as follows. With the aid of vaulting created by the scleral prosthesis 116, the upward movement of the ciliary muscles 112 actually pulls on the posterior zonules 114c and relaxes the anterior and equatorial zonules 114a-114b. This pulls upward on the posterior surface of the lens 103 (thereby causing it to “round up” and increase its effective dioptric power) and reduces or removes tension from the anterior surface of the lens 103 (thereby reducing or removing resistance to the lens “rounding-up”). This may change the position of the natural crystalline lens and/or increase the thickness of the lens 103. Increasing the thickness of the lens 103 increases the distance between the anterior surface and the posterior surface of the lens 103 in the center of the “visual axis,” thereby increasing the overall effective refractive power of the lens 103 and allowing the eye to focus on near objects clearly.

In FIG. 2, however, there is no scleral prosthesis 116. The upward movement of the ciliary muscles 112 might move the attachment point for the posterior zonules 114c up, but the posterior zonules 114c do not experience enough increase in tension to trigger the accommodative “rounding-up” of the lens 103. Thus, there may be little or no change in the shape or position of the lens 103, even though the anterior and equatorial zonules 114a-114b (which have relaxed) offer little or no resistance to a change in the shape of the lens 103. Again, however, regardless of the orientation of the zonules 114a-114c, it has been established that the insertion of one or more scleral prostheses 116 into a patient's eye can help to restore accommodative power to the crystalline lens 103 of the eye.

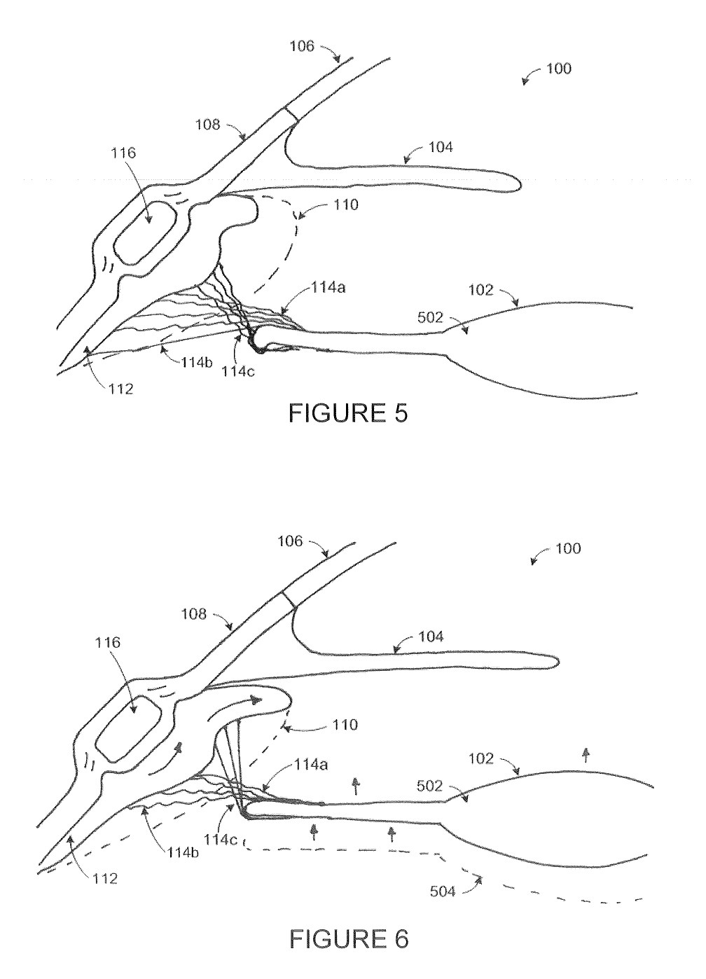

In accordance with this disclosure, the insertion of one or more scleral prostheses 116 into a patient's eye can also help to provide accommodative power to an artificial lens implanted into the patient's eye. FIGS. 5 and 6 illustrate an example eye 100 having a non-accommodating intraocular lens 502 focusing at distance and focusing at near with modification.

As shown in FIG. 5, the substance of the natural crystalline lens 103 has been entirely removed, leaving the zonules 114a-114c attached to the lens capsule 102 and the lens capsule 102. This could be done through a capsulorrhexis or small incision in the center of the anterior surface of the lens capsule. This could also involve the use of phacoemulsification, which includes using ultra-sound energy to break the natural crystalline lens 103 into very small pieces that can be vacuumed out through the use of suction instruments. The instruments used for capsulorrhexis and phacoemulsification could be inserted through a small incision (such as 2.7 mm) in the cornea 106 just above the limbus where the cornea 106 meets the sclera 108. This is often done to remove cataracts or to perform a Refractive Lens Exchange (RLE) to provide a solution for presbyopia. What remains after the natural lens 103 has been removed is the crystalline lens capsule 102 that formerly surrounded the lens 103, along with the zonular attachments (zonules 114a-114c) that helped to hold the lens 103 in place and still hold the lens capsule 102 in place.

A non-accommodating intraocular lens 502, such as one made from some form of acrylic, silicone or other material, is often (but not always) folded into an “injector” similar to a hypodermic needle. The injector is inserted through the small incision in the cornea 106 and through the incision made in the center of the lens capsule 102. The plunger on the injector is actuated, forcing the folded intraocular lens 502 out of the injector into the lens capsule 102 where it slowly unfolds. Eventually, the lens capsule 102 shrinks and forms itself to the shape of the particular intraocular lens 502. Nonetheless, the zonules 114a-114c remain attached to the lens capsule, such as 1.5-2.0 mm from the far edge of the intraocular lens 502. The intraocular lens 502 also includes “haptics” or small arms that are connected to the intraocular lens 502. The haptics help center the intraocular lens 502 in the lens capsule 102 so that the lens 502 remains directly in the optic axis.

In FIG. 5, the zonules 114a-114c are “wavy” to represent the fact that the person illustrated here is any patient that has had this type of surgery regardless of age and has therefore become presbyopic and (ii) because the eye is focused at distance. In the case of natural lens removal and replacement with a non-accommodating intraocular lens 502 as shown in FIG. 5, the zonules 114a-114c may be even more relaxed since the intraocular lens 502 does not have the same volume and shape as the natural lens 103 and thus the whole capsule may be looser. As in previous figures, the zonules 114a-114c may criss-cross as they attach to the ciliary muscles 112. Also, a scleral prosthesis 116 has been inserted into the patient's eye.

FIG. 6 illustrates the example eye 100 in a patient that has had this form of surgery regardless of age and has therefore become presbyopic, with the intraocular lens 502 focusing at near with modification. The arrangement shown in FIG. 6 is the same as that shown in FIG. 5, except that the eye is attempting to accommodate. Because of the presence of the scleral prosthesis 116, the zonules can exert more tension, and the intraocular lens 502 actually moves forward from its initial position 504 to the current position shown in FIG. 6. In this example, the ciliary muscle 112 moves up and in, which causes the posterior zonules 114c to tighten and pull the whole intraocular lens 502 up, vaulting the lens 502 forward. Because the distance between the anterior surface of the intraocular lens 502 and the cornea 104 has decreased, there is a vertex distance effect, increasing the effective dioptric power of the non-accommodating lens 502 and allowing the patient to see near objects more clearly.

In some embodiments, the non-accommodating lens 502 represents a monocular intraocular lens, meaning it has one fixed focal point. Without the scleral prosthesis 116, there has been no indication (by manufacturers or researchers) that there is even moderate improvement in near vision with normal non-accommodating monocular intraocular lenses as the ciliary muscles 112 attempt to accommodate. Near vision could often be improved with a normal monocular non-accommodating intraocular lens only if dioptric power (or “add”) is built into the prescription of the intraocular lens itself. Whatever focal length or near vision acuity is built into the prescription of the monocular intraocular lens is fixed once implanted in the patient and does not change. Conversely, even a normal monocular intraocular lens 502 can achieve some moderate to substantial accommodative effect due to vertex distance change if combined with the use of one or more scleral prostheses 116.

In other embodiments, multi-focal intraocular lenses 502 (such as refractive multi-focal intraocular lenses with concentric optic circles, diffractive multifocal lenses with concentric diffractive steps, or other aspheric designs allowing for both distance and near focal points with the same lens) could be used. A multi-focal intraocular lens 502 could be vaulted forward in a fashion similar to a normal monocular intraocular lens since their mechanical structures are very similar. This likewise may provide an accommodative effect as the ciliary muscles 112 contract in combination with one or more sclera prostheses 116. Example manufacturers of multi-focal intraocular lenses on the market today are ALCON (RESTORE) and AMO (REZOOM).

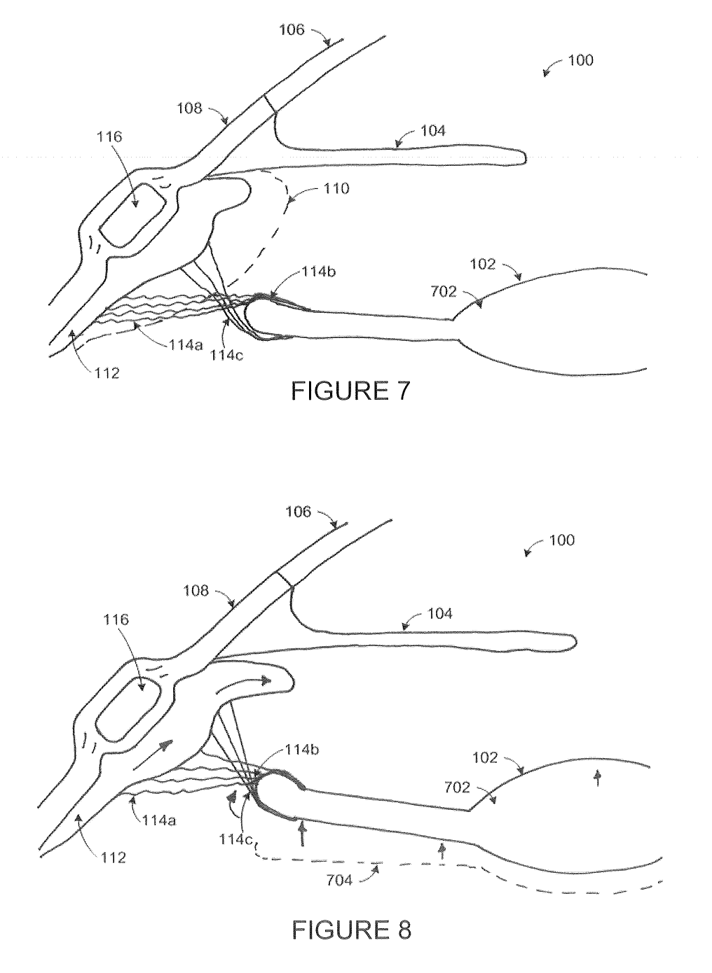

FIGS. 7 and 8 illustrate an example eye 100 having an accommodating intraocular lens 702 focusing at distance and focusing at near with modification. FIG. 7 is similar to FIG. 5. However, in this example, the accommodating intraocular lens 702 represents a single-optic accommodating lens, such as a CRYSTALENS intraocular lens by EYEONICS. In FIG. 7, the zonules 114a-114c are “wavy” because (i) the patient illustrated has had his or her natural lens replaced and is thus presbyopic regardless of age and (ii) the eye is focused at distance. While the haptics shown here are relatively flat, some versions of single-optic accommodating lens may have the haptics angled slightly downwards or slightly upwards towards the cornea. In other embodiments, the eye 100 in FIG. 7 could include an altered natural crystalline lens or a refilled natural lens capsule, which could have a shape more similar to the natural crystalline lens 103.

FIG. 8 illustrates the example eye 100 with the accommodating intraocular lens 702 focusing at near with modification. The arrangement shown in FIG. 8 is the same as that shown in FIG. 7, except that the eye 100 is attempting to accommodate and has vaulted the intraocular lens 702 (or the posterior portion of the altered natural crystalline lens or refilled natural lens capsule) forward. In this example, the posterior zonules 114c have moved forward and are tightened due to the presence of the scleral prosthesis 116. Also, in some embodiments, the whole lens not only vaults forward, but the CRYSTALENS haptics (which are designed to bend at “hinges” where the haptics are attached to the lens optics) or other lens haptics also bend forward from the hinges. This could cause some form of arching in the anterior surface of the single lens optic, increasing its refractive power (in addition to the whole lens being closer to the cornea 104, which also provides a vertex distance effect). As such, by increasing the amount of bend in the CRYSTALENS or other haptics and by providing more forward vaulting movement, the scleral prosthesis 116 can substantially improve the current performance of the CRYSTALENS accommodating lens or any other accommodating lenses that have a similar structure or mode of action.

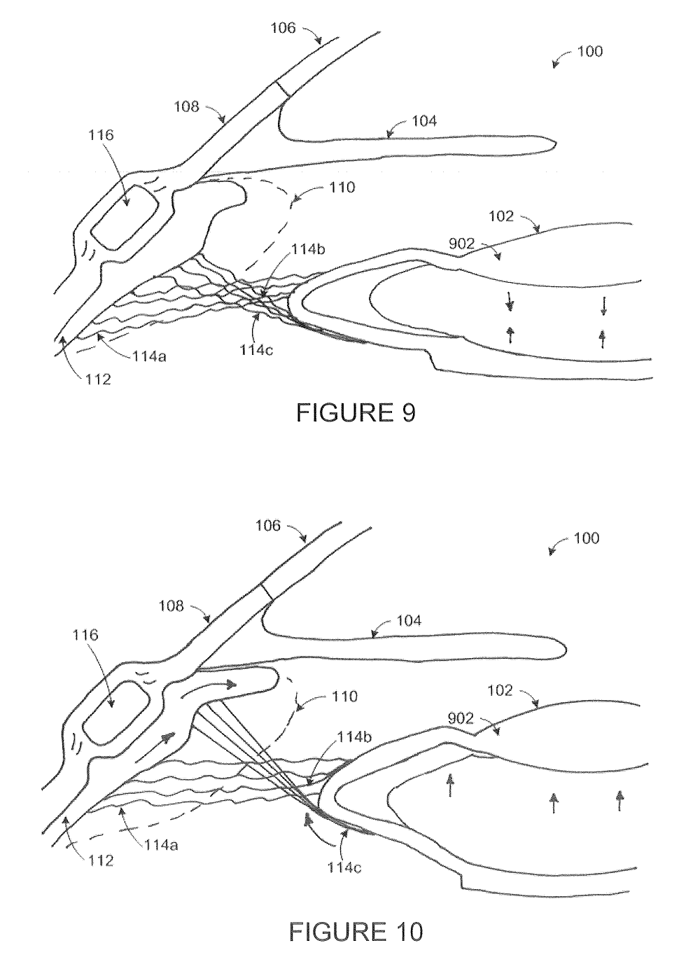

FIGS. 9 and 10 illustrate an example eye 100 having another accommodating intraocular lens 902 focusing at distance and focusing at near with modification. In FIG. 9, the zonules 114a-114c are again “wavy” because (i) the patient illustrated here has had his or her natural lens replaced and is thus presbyopic regardless of age and (ii) the eye is focused at distance. In other embodiments, the eye 100 in FIG. 9 could include an altered natural crystalline lens or a refilled natural lens capsule, which could also have a shape more similar to the natural crystalline lens 103.

In this example, the intraocular lens 902 represents a dual-optic accommodating lens or any other lens that changes its effective dioptric power through mechanical, hydraulic, laser, electrical, refractive index manipulation, chemical, or other means. As a particular example, the intraocular lens 902 could represent an intraocular lens by VISIOGEN. This type of lens could have approximately the same volume and dimensions as the natural crystalline lens 103. In particular embodiments, there may be a “negative” lens on the posterior side of the intraocular lens 902 and a very high-power “positive” lens on the anterior side of the intraocular lens 902. When these get farther apart, there is an increase in near vision magnification, allowing a patient to focus on near objects more clearly.

The VISIOGEN lens design (or other similar designs) creates manufactured tension at the rounded edges where the zonules 114a-114c attach, acting somewhat like a spring to allow the lens 902 to expand and increase its refractive power. The inward pointing arrows in FIG. 9 indicate that some force is necessary to keep the anterior and posterior lenses relatively close to each other to allow the eye to see properly at distance. However, if the tension at the edges allows the lens 902 to expand prematurely, the patient may actually lose distance vision, constantly focusing at near even when the patient does not want to. The same can be true for an altered natural crystalline lens or a refilled natural crystalline lens capsule. In a presbyopic eye where the tension on the zonules 114a-114c has been lost with age, it is not completely clear what creates the tension on the zonules to keep the VISIOGEN lens in its flattened, non-accommodated position. However, as with the natural crystalline lens in a presbyopic eye, there may be enough residual tension with the zonules 114a-114c to maintain equal tension on the zonules, keeping the lens focused at distance. Correspondingly, for sake of illustration, the lens 902 has been drawn in its flattened position in FIG. 9.

FIG. 10 illustrates the eye 100 with the accommodating intraocular lens 902 focusing at near with modification. As shown here, the ciliary muscles 112 contract and move upward and inward. This movement and the presence of the scleral prosthesis 116 help to move the attachment point for the posterior zonules 114c up, which increases the tension on the posterior zonules 114c and triggers the accommodative response of the lens 902 (allowing it to “round-up”). At the same time, the upward motion of the ciliary muscle 112 relaxes the anterior and equatorial zonules 114a-114b, reducing or removing tension from the anterior surface of the lens 902 and thereby reducing or removing resistance to the lens “rounding-up”.

As mentioned above, the increased distance between the anterior surface and the posterior surface of the lens 902 in the center of the “visual axis” increases the overall refractive power of the lens 902, allowing the eye to focus on near objects clearly. The presence of the scleral prosthesis 116 could help to improve the performance of the lens 902 or any other dual-optic or multi-optic accommodating intraocular lens or any lens (artificial or natural) that changes its effective dioptric power through mechanical, hydraulic, laser, electrical, refractive index manipulation, chemical, or any other means. The presence of the scleral prosthesis 116 could also help to improve the performance of an altered natural crystalline lens or a refilled natural crystalline lens capsule by restoring the natural tension on the zonules 114a-114c at near.

To summarize, one or more scleral prostheses 116 can be used beneficially with various types of intraocular lenses, altered natural crystalline lenses, or refilled natural crystalline lens capsules. For example, the scleral prostheses 116 could be used with any accommodating intraocular lens, altered natural crystalline lens, or refilled natural crystalline lens capsule to improve the natural triggering mechanism for accommodation in the eye, thereby helping to improve the performance of the accommodating lens, altered natural crystalline lens, or refilled natural crystalline lens capsule. The scleral prostheses 116 could also be used with any non-accommodating lens to vault the lens forward and provide an increase in dioptric power due to the vertex distance effect. There are many accommodating and non-accommodating intraocular lens designs currently on the market or in development (some with very complex mechanics) that could be coupled with the scleral prostheses 116.

While the use of scleral prostheses 116 in conjunction with intraocular lenses, altered natural crystalline lenses, or refilled natural crystalline lens capsules have been described above, other techniques could also be used to increase the effectiveness of intraocular lenses. For example, as noted in various ones of the U.S. patent documents incorporated by reference above, it is possible to perform laser ablations (or other laser techniques) to remove portions of the scleral tissue from an eye, which allows the sclera in those areas to have an altered rigidity and to possibly expand and increase the diameter of sclera over the ciliary muscles 112. It is also possible to fill in the ablation or other area with a collagen block, collagen shield, or other component to prevent healing and to keep the increased scleral volume intact. This type of technique could also be used in conjunction with intraocular lenses to provide increased accommodation. In fact, any suitable technique for increasing accommodation through scleral expansion, scleral manipulation, scleral relaxation, or other mechanisms could be used in conjunction with an artificial intraocular lens, altered natural crystalline lens, or refilled natural crystalline lens capsule.

Moreover, recent research indicates that the posterior zonules 114c in the eye may attach to the Hyaloid membrane, which separates the posterior chamber of the eye (filled with aqueous) from the vitreous cavity of the eye (filled with vitreous). The posterior zonules 114c may then continue along the surface of the Hyaloid membrane and down the posterior surface of the lens capsule 103 to their ultimate attachment points further down the capsule. In some embodiments, the zonules' attachment to the Hyaloid membrane can be used to enlist the strength and total coverage of the Hyaloid membrane itself to pull-up the attachment points for the posterior zonules 114c, triggering accommodation.

In addition, the movement of the ciliary muscles 112 during accommodation and the arrangement/orientation of the zonules 114a-114c shown in the figures above are based on recent research. However, the actual movement of the ciliary muscles 112 during accommodation and the actual arrangement/orientation of the zonules 114a-114c remain subject to further research (by both the assignee referenced above and others in the field) and may eventually be shown to be different than that shown above. Even if it is unclear how the precise mechanics of the eye operate in conjunction with one or more scleral prostheses 116 and an artificial intraocular lens, altered natural crystalline lens, or refilled natural crystalline lens capsule, it can be shown that the presence of one or more scleral prostheses 116 in the eye can help to improve the effectiveness of the artificial intraocular lens, altered natural crystalline lens, or refilled natural crystalline lens capsule. This improved effectiveness could take the form of providing accommodation to a non-accommodating IOL or by improving accommodation of an accommodating IOL, altered natural crystalline lens, or refilled natural crystalline lens capsule.

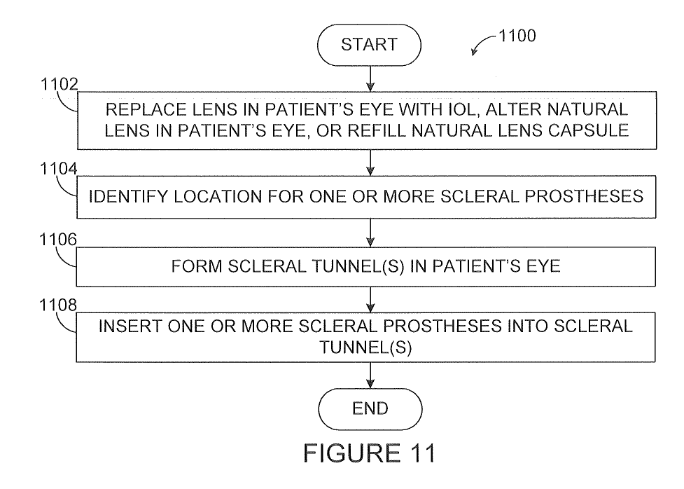

FIG. 11 illustrates an example method 1100 for providing improved accommodation in an eye. As shown in FIG. 11, the natural lens in a patient's eye is replaced with an IOL, the natural lens is modified, or the natural lens capsule is refilled at step 1102. This could include, for example, removing the natural lens 103 and inserting an accommodating or non-accommodating intraocular lens in the patient's eye. This could also include using any suitable technique to alter the natural lens, including (but not limited to) pharmaceutical agents, lasers, electromagnetic waves, magnetic waves, and/or sound or ultrasound. As a particular example, this could include softening the lens, such as by using laser irradiation. This could further include using any suitable lens capsule refilling technique. At this point, the patient may have little to no accommodative ability in the eye.

A location for one or more scleral prostheses is determined at step 1104, one or more scleral tunnels are formed in the patient's eye at step 1106, and one or more scleral prostheses are inserted into the one or more scleral tunnels at step 1108. Various tools and techniques for identifying a location for a scleral prosthesis are disclosed in the U.S. patent documents incorporated by reference above. Also, various tools and techniques for forming a scleral tunnel are disclosed in the U.S. patent documents incorporated by reference above. In addition, various scleral prostheses are disclosed in the U.S. patent documents incorporated by reference above. The one or more scleral prostheses can be used to provide accommodative abilities to a non-accommodating IOL. The one or more scleral prostheses can also be used to improve the accommodative abilities of an accommodating IOL, a modified natural lens, or a refilled lens capsule.

Although FIG. 11 illustrates an example method 1100 for providing improved accommodation in an eye, various changes may be made to FIG. 11. For example, the insertion of the IOL, the modification to the lens, or the refilling of the lens capsule may or may not occur at the same time that the one or more scleral prostheses are inserted into the eye.

It may be advantageous to set forth definitions of certain words and phrases used throughout this patent document. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A system comprising:
an intraocular lens configured to replace a natural crystalline lens of an eye; and
one or more scleral prostheses configured to be inserted into scleral tissue of the eye, the one or more scleral prostheses configured to modify a structure of the eye to improve an accommodative ability of the eye with the intraocular lens.

2. The system of claim 1, wherein:
the intraocular lens comprises an accommodating intraocular lens; and
the one or more scleral prostheses are configured to increase an amount of accommodation achievable using the accommodating intraocular lens.

3. The system of claim 2, wherein the accommodating intraocular lens comprises a single-optic accommodating lens.

4. The system of claim 2, wherein the accommodating intraocular lens comprises a multi-optic accommodating lens.

5. The system of claim 4, wherein the multi-optic accommodating lens has a shape similar to a shape of the natural crystalline lens of the eye.

6. The system of claim 1, wherein:
the intraocular lens comprises a non-accommodating intraocular lens; and
the one or more scleral prostheses are configured to provide an amount of accommodation achievable using the non-accommodating intraocular lens.

7. The system of claim 1, wherein the system comprises multiple scleral prostheses.

8. A method comprising:
inserting an intraocular lens into an eye to replace a natural crystalline lens of the eye; and
inserting one or more scleral prostheses into scleral tissue of the eye, the one or more scleral prostheses modifying a structure of the eye and improving an accommodative ability of the eye with the intraocular lens.

9. The method of claim 8, wherein:
the intraocular lens comprises an accommodating intraocular lens; and
the one or more scleral prostheses improve an amount of accommodation achievable using the accommodating intraocular lens.

10. The method of claim 9, wherein the accommodating intraocular lens comprises a single-optic accommodating lens.

11. The method of claim 9, wherein the accommodating intraocular lens comprises a multi-optic accommodating lens.

12. The method of claim 8, wherein:
the intraocular lens comprises a non-accommodating intraocular lens; and
the one or more scleral prostheses provide an amount of accommodation achievable using the non-accommodating intraocular lens.

13. A method comprising:
modifying a natural lens of an eye; and
inserting one or more scleral prostheses into scleral tissue of the eye, the one or more scleral prostheses modifying a structure of the eye to improve an accommodative ability of the eye with the modified natural lens.

14. The method of claim 13, wherein modifying the natural lens comprises softening the natural lens using a laser.

15. The method of claim 13, wherein modifying the natural lens comprises modifying the natural lens using at least one of: a pharmaceutical agent, a laser, electromagnetic waves, magnetic waves, sound, and ultrasound.

16. The method of claim 13, wherein inserting one or more scleral prostheses into the scleral tissue of the eye increases tension on zonules of the eye, thereby improving the accommodative ability of the eye.

17. The method of claim 13, wherein inserting one or more scleral prostheses into the scleral tissue of the eye comprises inserting multiple scleral prostheses into the scleral tissue of the eye.

18. A method comprising:
filling a crystalline lens capsule of an eye with one or more materials; and
inserting one or more scleral prostheses into scleral tissue of the eye, the one or more scleral prostheses modifying a structure of the eye to improve an accommodative ability of the eye with the filled crystalline lens capsule.

19. The method of claim 18, wherein inserting one or more scleral prostheses into the scleral tissue of the eye increases tension on zonules of the eye, thereby improving the accommodative ability of the eye.

20. The method of claim 18, wherein inserting one or more scleral prostheses into the scleral tissue of the eye comprises inserting multiple scleral prostheses into the scleral tissue of the eye.
